(19) United States
(12) Patent Application Publication
(10) Pub. No.: US 2025/0120778 A1
(43) Pub. Date: Apr. 17, 2025

(54) AUTOMATIC HANDLE ASSIGNMENT IN SURGICAL ROBOTIC SYSTEM

(71) Applicant: Covidien LP, Mansfield, MA (US)

(72) Inventors: Paul M. Loschak, Somerville, MA (US); Dan Liu, Beachwood, OH (US); Walter Schoen, Cambridge, MA (US); Meredith E. Koch, East Walpole, MA (US); Kevin R. Fogarty, New York, NY (US)

(21) Appl. No.: 18/294,756

(22) PCT Filed: Sep. 22, 2022
(86) PCT No.: PCT/IB2022/058987

§ 371 Date: Feb. 2, 2024

Related U.S. Application Data
(60) Provisional application No. 63/247,951, filed on Sep. 24, 2021.

(51) Int. Cl. A61B 34/37; A61B 34/00; A61B 34/20; A61B 50/13

(52) U.S. Cl. CPC A61B 34/37; A61B 34/25; A61B 34/74; A61B 50/13; A61B 2034/2055

(57) ABSTRACT

A surgical robotic system having a plurality of movable carts each having a robotic arm holding a surgical instrument is configured to automatically assign each of the instruments to a designated region on a graphical user interface. This corresponds the instruments on the graphical user interface to the location of the movable carts around a surgical table, allowing for easy identification of the instruments by a surgeon.

### BACKGROUND

Surgical robotic systems may include a surgical console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body.

Prior to commencing a surgical procedure, the surgical robotic systems are configured, such as assigning surgical robotic arms to handle controllers. This process is performed by the surgeon or a technician, which may be time consuming and error prone due to manual inputs.

### SUMMARY

According to one embodiment of the present disclosure, a surgical robotic system is disclosed. The surgical robotic system includes a plurality of movable carts each of the movable carts may include a robotic arm. The plurality of movable carts includes a camera movable cart having a camera and instrument movable carts, each of which may include a surgical instrument. The system also includes a surgeon console having a display configured to display a graphical user interface with a plurality of graphical representations each of which corresponds to one instrument movable cart. The system further includes a controller configured to assign each of the instrument movable carts to one graphical representation based on orientation of the instrument movable carts.

Implementations of the above embodiment may include one or more of the following features. According to one aspect of the above embodiment, the surgeon console may further include a left-hand controller and a right-hand controller, where each of the left-hand controller and the right-hand controller is configured to control one selected surgical instrument of the surgical instruments. A first portion of the plurality of graphical representations may be assigned to the left-hand controller and a second portion of the plurality of graphical representations may be assigned to the right-hand controller. The controller may be further configured to assign each of the instrument movable carts to one graphical representation based on an orientation of the camera. The controller may be further configured to assign each of the instrument movable carts to one graphical representation based on an angle of each of the instrument movable carts relative to a surgical table. The display may be a touchscreen and each graphical representation of the plurality of graphical representations is movable on the touchscreen.

According to another embodiment of the present disclosure, a surgical robotic system is disclosed. The surgical robotic system includes a plurality of movable carts each of the movable carts including a robotic arm. The plurality of movable carts may include a camera movable cart having a camera and instrument movable carts, each of which may include a surgical instrument. The system may include a display configured to display a graphical user interface having a plurality of graphical representations each of which corresponds to one instrument movable cart. The system also includes a controller configured to assign each of the instrument movable carts to one graphical representation based on orientation of the instrument movable carts.

Implementations of the above embodiment may include one or more of the following features. According to one aspect of the above embodiment, the surgical robotic system may include a surgeon console having a left-hand controller and a right-hand controller, where each of the left-hand controller and the right-hand controller is configured to control one selected surgical instrument of the surgical instruments. A first portion of the plurality of graphical representations may be assigned to the left-hand controller and a second portion of the plurality of graphical representations may be assigned to the right-hand controller. The controller may be further configured to assign each of the instrument movable carts to one graphical representation based on an orientation of the camera. The controller may be further configured to assign each of the instrument movable carts to one graphical representation based on an angle of each of the instrument movable carts relative to a surgical table. The display may be a touchscreen and each graphical representation of the plurality of graphical representations is movable on the touchscreen.

According to a further embodiment of the present disclosure, a method for controlling a surgical robotic system is disclosed. The method may include receiving cart orientation data for each instrument movable cart of a plurality of instrument movable carts; displaying a graphical user interface having a plurality of graphical representations each of which corresponds to one instrument movable cart. The method may also include assigning each of the instrument movable carts to one graphical representation based on orientation data of the instrument movable carts.

Implementations of the above embodiment may include one or more of the following features. According to one aspect of the above embodiment, the method may include assigning a first portion of the plurality of graphical representations to a left-hand controller. The method may also include assigning a second portion of the plurality of graphical representations to a right-hand controller. The method may further include receiving camera orientation data for a camera held by a camera movable cart. The method may also include assigning each of the instrument movable carts to one graphical representation based on the camera orientation data. Orientation data of the instrument movable carts may include an angle of each of the instrument movable carts relative to a surgical table. The method may further include moving at least one graphical representation of the plurality of graphical representations on the graphical user interface.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a movable cart according to an aspect of the present disclosure;

FIG. 2 is a perspective view of a surgical robotic arm of the surgical robotic system of FIG. 1 according to an aspect of the present disclosure;

FIG. 3 is a perspective view of a setup arm with the surgical robotic arm of the surgical robotic system of FIG. 1 according to an aspect of the present disclosure;

FIG. 4 is a schematic diagram of a computer architecture of the surgical robotic system of FIG. 1 according to an aspect of the present disclosure;

FIG. 5 is a plan schematic view of movable carts of FIG. 1 positioned about a surgical table according to an aspect of the present disclosure;

FIG. 6 is a graphical user interface displayed on a display of the surgeon console according to an embodiment of the present disclosure; and

FIG. 7 is a flow chart of a method for configuring the surgical robotic system of FIG. 1 according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views. As used herein the term “distal” refers to the portion of the surgical robotic system and/or the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, a personal computer, or a server system.

As will be described in detail below, the present disclosure is directed to a surgical robotic system, which includes a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

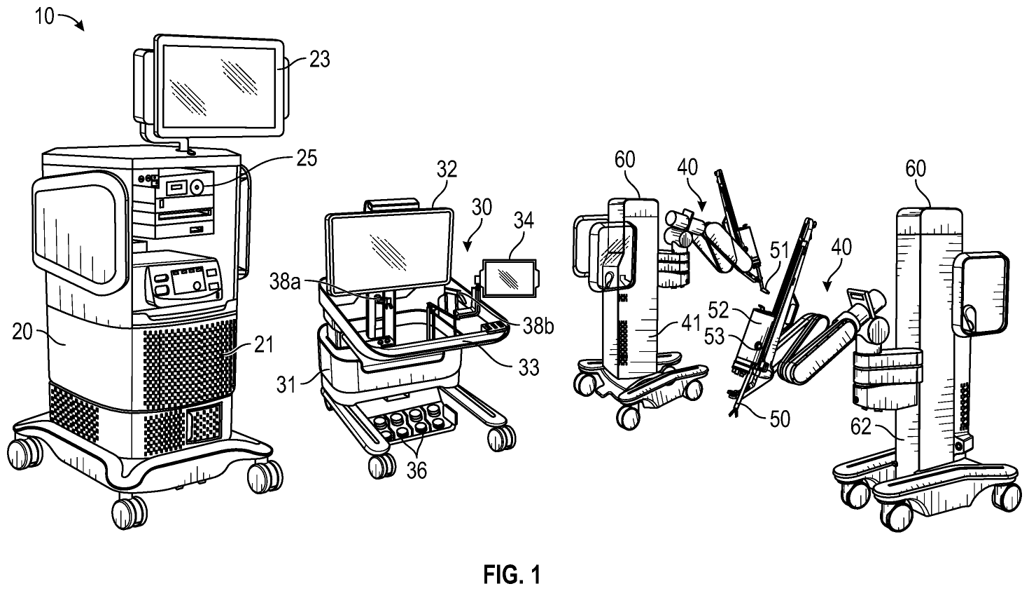

With reference to FIG. 1, a surgical robotic system 10 includes a control tower 20, which is connected to all of the components of the surgical robotic system 10 including a surgical console 30 and one or more movable carts 60. Each of the movable carts 60 includes a robotic arms 40 having a surgical instrument 50 removably coupled thereto. The robotic arms 40 is also coupled to the movable cart 60.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the present disclosure and output the processed video stream.

The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

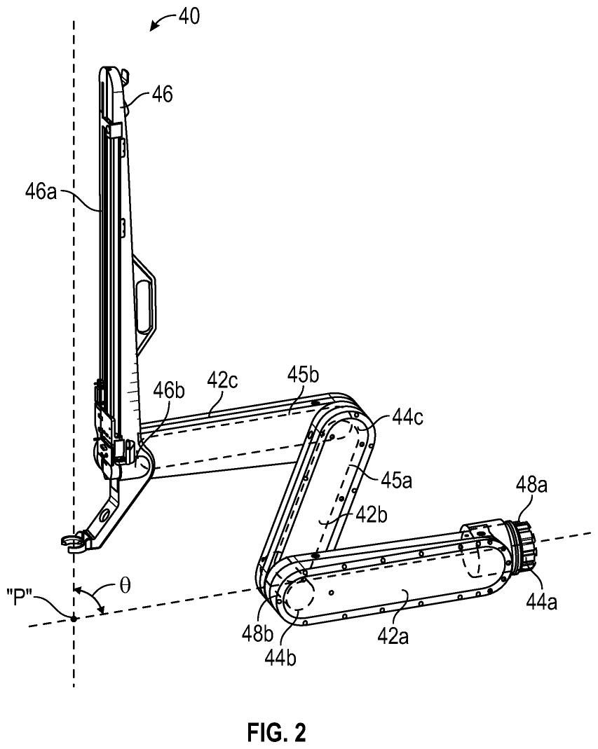

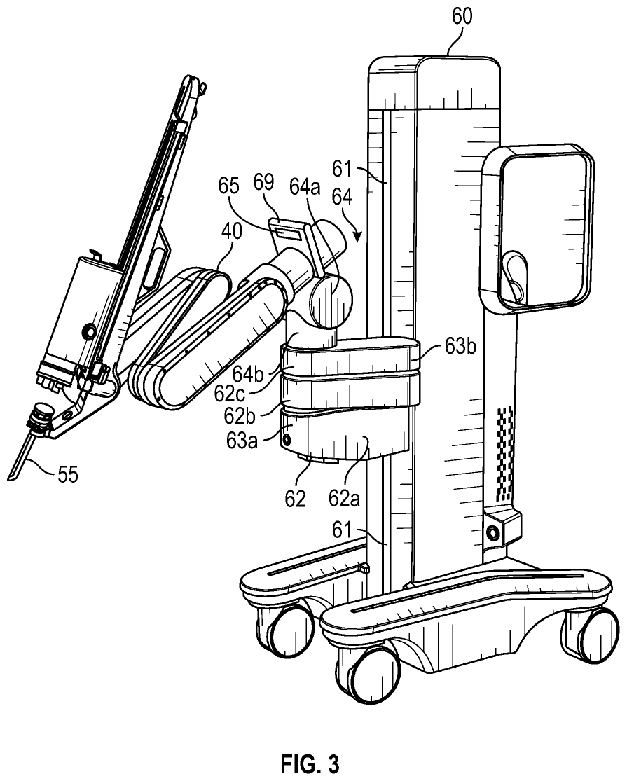

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46.

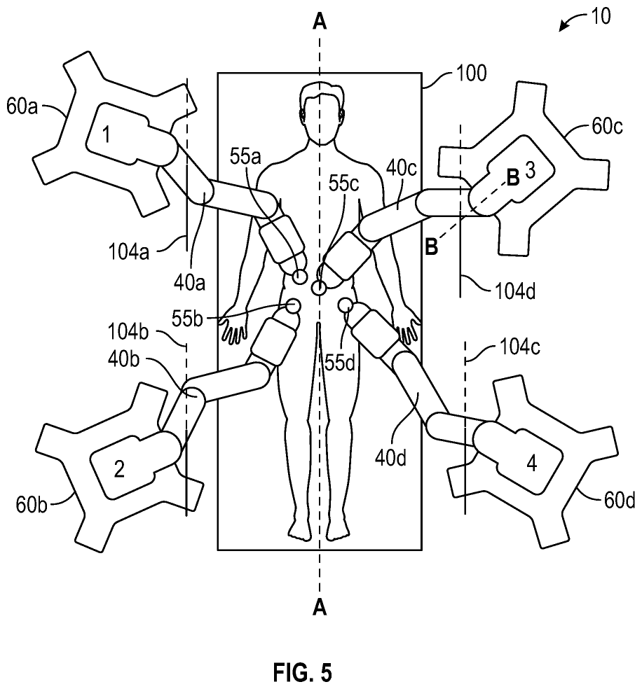

The robotic arm 40 also includes a plurality of manual override buttons 53 (FIGS. 1 and 5) disposed on the IDU 52 and the setup arm 62, which may be used in a manual mode. The user may press one or more of the buttons 53 to move the component associated with the button 53.

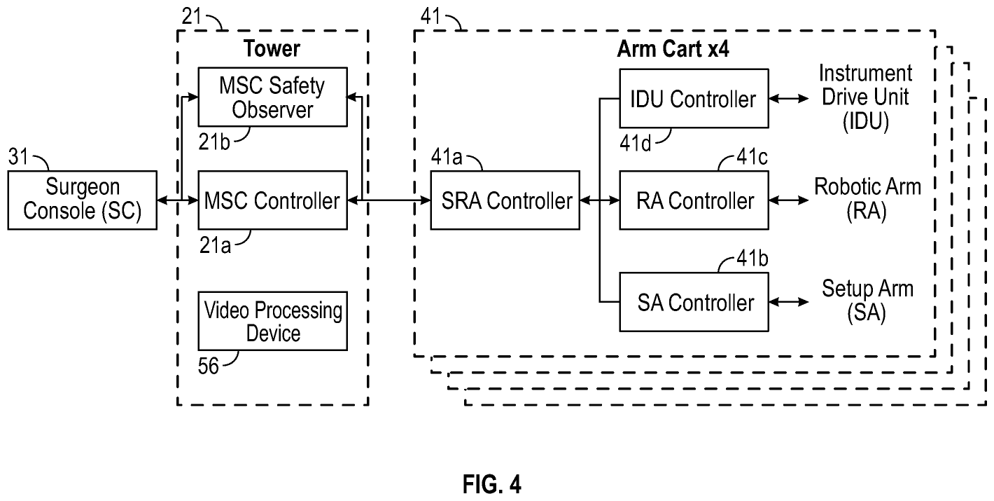

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In embodiments, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

With reference to FIG. 5, the surgical robotic system 10 is setup around the surgical table 100. The system 10 includes movable carts 60a-d, which may be numbered “1” through “4.” The movable carts 60a-d may be positioned relative to the surgical table 100 and each other using any suitable registration system or method. During setup, each of the carts 60a-d are positioned around the surgical table 100. Position and orientation of the carts 60a-d depends on a plurality of factors, such as placement of a plurality of ports 55a-d, which in turn, depends on the procedure being performed. Once the port placement is determined, the ports 55a-d are inserted into the patient, and carts 60a-d are positioned to insert instruments 50 and the endoscopic camera 51 into corresponding ports 55a-d. Orientation of the carts 60a-d and their corresponding robotic arms 40a-d may be based on individual laser alignment patterns 104a-d. For a more detailed description of using alignment patterns to orient a plurality of movable carts 60 see International Application No. PCT/US2021/034125, titled “SURGICAL ROBOTIC SYSTEM USER INTERFACES”, filed on May 26, 2021, the entire disclosure of which is incorporated by reference herein.

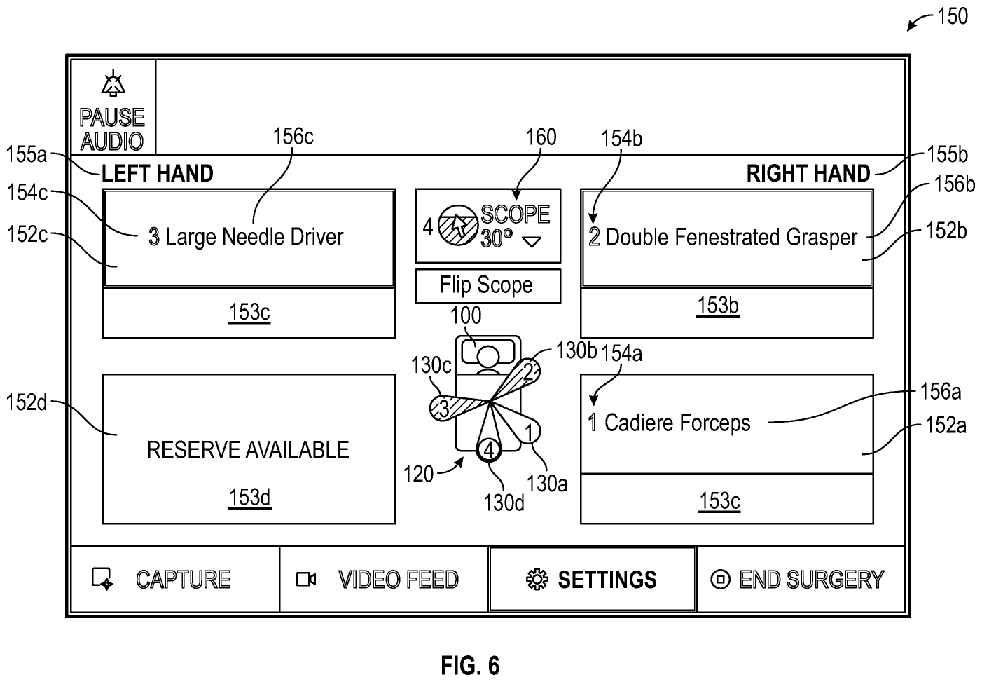

With reference to FIG. 6, a GUI 150 is displayed on the second display 34 of the surgical console 30 and/or the display 23 of the control tower 20. Once the movable carts 60a-d are placed at their desired positions, the surgeon or any other personnel registers each of the robotic arms 40a-d on a graphical user interface (GUI) 150, which may be displayed on the second display 34 or any other display of the surgical robotic system 10, e.g., displays 23 or 32.

The GUI 150 includes a plurality of regions 153a-d which include graphical representations 152a-c for each of the three robotic arms 40a-c numbered “1”-“3” and a reserve graphical representation 152d. Each of the graphical representations 152a-c includes an identification number 154a-c and an instrument type 156a-c. The GUI 150 also includes a region 160. The region 160 shows an arm identification number 154d and an orientation indicator for the indicator including pitch angle of the camera 51 and rotation relative to a horizontal plane. The region 160 also shows that the camera 51 is coupled to the robotic arm 40d numbered “4”. A fourth region 153d is reserved for reassigning any one of the graphical representations 152a-c. Similarly, the third region 153c may also be a placeholder.

The GUI 150 also shows a bed map 120 having the surgical table 100 and each of the robotic arms 40a-d represented as arrows 130a-d. The bed map 120 allows the users to quickly recognize the relationship of the corresponding movable carts 60a-d to the surgical table 100. Each of the arrows 130a-d may display information pertaining to each of the corresponding movable carts 60a-d, such as an arm identification number, namely “1”-“4”, registered yaw angle, etc. The angle may be input manually or based on a laser alignment using the laser alignment patterns 104a-d for each of the corresponding movable carts 60a-d.

The movable carts 60a-d may be automatically assigned to each of the graphical representations 152a-c, with the graphical representations 152a and 152b being controlled by the right-hand controller 38b and the graphical representations 152c and 152d being controlled by the left-hand controller 38a. However, the surgeon may move the instruments 50, i.e., robotic arms 40a-c between any of the four graphical representations 152a-d.

As noted above, the second display 34 is a touchscreen, which allows for moving the graphical representations 152a-d between the regions 153a-d by pressing, holding, and moving or using any other suitable touch gestures, e.g., moving the graphical representation 152a from the region 153a to any of the other regions 153b-d. This assigns the instrument to a desired one of the hand controllers 38a and 38b, designated as a left-hand column 155a and a right-hand column 155b, respectively. As the icons are moved between any of the graphical representations 152a-c, the user can confirm the actual physical location of the instruments 50 and their corresponding robotic arms 40a-d by matching the colors displayed on the GUI 150 to the colors on the color indicators 102a-d regardless of which graphical representation 152a-d is being used.

The controller 21a automatically assigns the movable carts 60a-d and corresponding instruments 50a-c to the regions 153a-c of the GUI 150. In embodiments, the controller 21a may assign instrument movable carts 60a-c in numerical order, based on the number, i.e., 1-3, of the movable carts 60a-c such that the first arm cart 60a numbered “1” is assigned to the first region 153a, the second arm cart 60b numbered “2” is assigned to the second region 153b, and the third arm cart 60c numbered “3” is assigned to the third region 153c, with the fourth region 153d being held in reserve. However, occasionally instrument movable carts 60a-c are positioned on one side (e.g., right) of the surgical table 100 but are automatically assigned to the opposite side handle controller 38a (e.g., left) due to the numbering of the instrument movable carts 60a-c. In embodiments, once the automatic assignment is completed the surgeon or the technician may manually move the graphical representation 152a-c to any of the regions 153a-d to correlate correct position of the movable carts 60a-c to the regions 153a-d.

In addition to manual assignment of the graphical representation 152a-c of the instrument movable carts 60a-c to the regions 153a-d, the present disclosure also provides an algorithm to automatically assign the graphical representation 152a-c to the correct region of the regions 153a-d. The algorithm may be embodied as software instructions executed by the controller 21a.

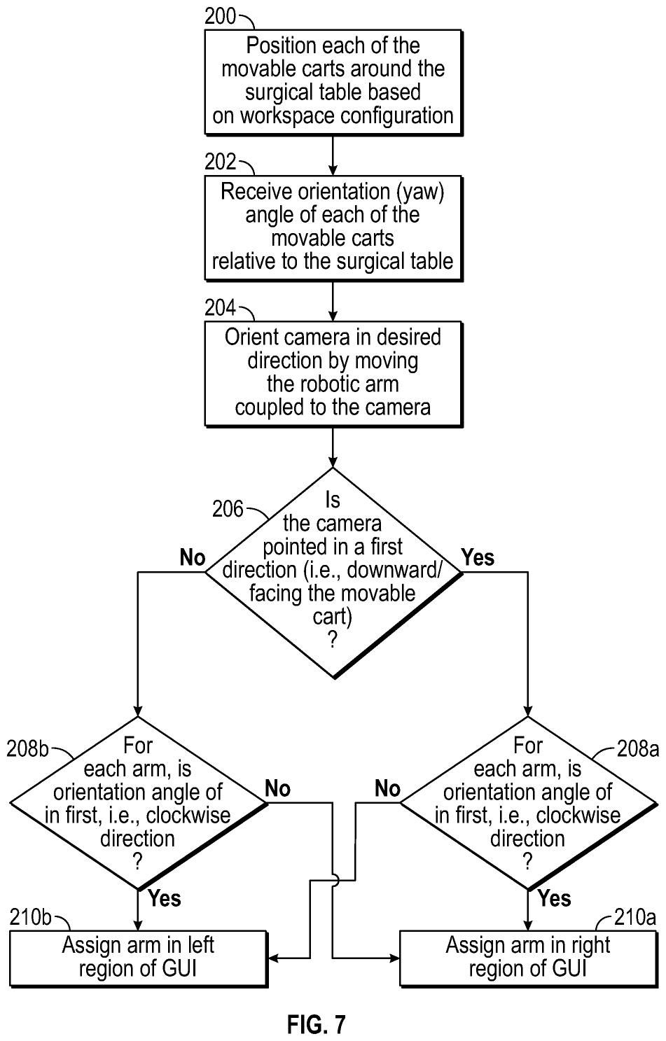

With reference to FIG. 7, a method for automatically assigning graphical representations 152a-c of instruments 50a-c to regions 153a-d includes at step 200 positioning the movable carts 60a-d around the surgical table 100. Positioning of the movable carts 60a-d is based on placement of the ports 55a-d into the patient, which in turn, is based on workspace requirements of the surgical procedure.

At step 202, once the movable carts 60a-d are positioned around the surgical table 100, the yaw angle of each of the movable carts 60a-d is provided to the surgical system 10, i.e., the controller 21a of the computer 21. The yaw angle may be calculated relative to a longitudinal axis “A-A” bisecting the surgical table 100. The angle may be calculated using the patterns 104a-d, which are initially projected in alignment with a longitudinal axis “B-B” of each of the movable carts 60a-d and then rotated to align with the longitudinal axis “A-A”. The rotation angle as well as direction to achieve alignment provides the yaw angle for each of the movable carts 60a-d. Rotation angle and/or direction may be expressed from 0-360° or 0-180° to include direction of rotation. Direction may be either in a first arm orientation direction, i.e., clockwise, or a second arm orientation direction, i.e., counterclockwise.

At step 204, the instruments 50a-c are inserted into the respective ports 55a-c and the camera 51 is inserted into the port 55d. Thereafter, the robotic arm 40 holding the camera 51 is moved to orient the camera 51 in a desired direction. The camera 51 may be pointed in a first camera direction, i.e., downward and/or toward the movable cart 60d supporting the camera 51, i.e., via the robotic arm 40, or the camera 51 may be pointed in a second camera direction, i.e., upward and/or away from the movable cart 60d.

Once the movable carts 60a-d are positioned and the instruments 50a-c as well as the camera 51 are inserted and oriented at their desired location and/or direction, the controller 21a populates the GUI 150 based on the direction of the camera 51 and the orientation of each of the movable carts 60a-d. The camera 51 and the corresponding movable cart 60d is assigned to the region 160. At step 206, the controller 21a determines whether the camera 51 is pointing in the first or second camera direction based on the configuration of the robotic arm 40 supporting the camera 51. Thereafter at step 208a and 208b, the controller 21a determines whether each of the remaining carts the movable carts 60a-c is oriented in the first arm direction or the second arm direction.

If the camera 51 is oriented in the first camera direction and one of the movable carts 60a-c is oriented in the first arm direction, the controller 21a in step 210a assigns that instrument 50 to the right-hand column 155b. In addition, the controller 21a displays a corresponding graphical representation 152a-c of the instrument on the regions 153b or 153c. Conversely, if the camera 51 is oriented in the first direction and one of the movable carts 60a-c is oriented in the second arm direction, in step 210b the controller 21a assigns that instrument 50 to the left-hand column 155a. The controller 21a also displays a corresponding graphical representation 152a-c of the instrument on the regions 153a or 153d.

The algorithm accounts for the change in orientation of the camera 51 by shifting the arrangement of the movable carts 60a-d with the perspective of the camera 51. Thus, if the camera 51 is oriented in the second camera direction and one of the movable carts 60a-c is oriented in the first arm direction, the controller 21a determines that the movable cart 60a-c is on the right side of the surgical table 100, in step 210a assigns that instrument 50 to the left-hand column 155a. The controller 21a also displays a corresponding graphical representation 152a-c of the instrument on the regions 153a or 153d. Conversely, if the camera 51 is oriented in the second direction and one of the movable carts 60a-c is oriented in the second arm direction, in step 210a the controller 21a assigns that instrument 50 to the right-hand column 155b. In addition, the controller 21a displays a corresponding graphical representation 152a-c of the instrument on the regions 153b or 153c.

If more than one instruments 50a-c is present on one side, then the instrument 50 whose movable cart 60 is closest to the top, i.e., head, of the surgical table 100 is assigned to the top region 153a or 153b. The instrument 50 that is furthest from the top is assigned to one of the bottom regions 153d or 153c. Distance from the top of the surgical table 100 may be determined using the yaw angle of the movable cart 60. In particular, the smaller the angle, e.g., 0-180° the closer the movable cart 60 is to the top of the surgical table 100.

After the controller 21a determines orientation of each of the movable carts 60a-c relative to the surgical table 100, the controller 21 a then populates each of the regions 153a-d with the name of the instruments 50a-c leaving any unfilled regions 153a-d as reserved.

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a plurality of movable carts each of the movable carts including a robotic arm, the plurality of movable carts includes a camera movable cart having a camera and instrument movable carts, each of which includes a surgical instrument;
a surgeon console including a display configured to display a graphical user interface having a plurality of graphical representations each of which corresponds to one instrument movable cart; and
a controller configured to assign each of the instrument movable carts to one graphical representation based on orientation of the instrument movable carts.

2. The surgical robotic system according to claim 1, wherein the surgeon console further includes a left-hand controller and a right-hand controller, wherein each of the left-hand controller and the right-hand controller is configured to control one selected surgical instrument of the surgical instruments.

3. The surgical robotic system according to claim 2, wherein a first portion of the plurality of graphical representations are assigned to the left-hand controller and a second portion of the plurality of graphical representations are assigned to the right-hand controller.

4. The surgical robotic system according to claim 1, wherein the controller is further configured to assign each of the instrument movable carts to one graphical representation based on an orientation of the camera.

5. The surgical robotic system according to claim 1, wherein the controller is further configured to assign each of the instrument movable carts to one graphical representation based on an angle of each of the instrument movable carts relative to a surgical table.

6. The surgical robotic system according to claim 1, wherein the display is a touchscreen and each graphical representation of the plurality of graphical representations is movable on the touchscreen.

7. A surgical robotic system comprising:
a plurality of movable carts each of the movable carts including a robotic arm, the plurality of movable carts includes a camera movable cart having a camera and instrument movable carts, each of which includes a surgical instrument;
a display configured to display a graphical user interface having a plurality of graphical representations each of which corresponds to one instrument movable cart; and
a controller configured to assign each of the instrument movable carts to one graphical representation based on orientation of the instrument movable carts.

8. The surgical robotic system according to claim 7, further comprising:
a surgeon console including a left-hand controller and a right-hand controller, wherein each of the left-hand controller and the right-hand controller is configured to control one selected surgical instrument of the surgical instruments.

9. The surgical robotic system according to claim 8, wherein a first portion of the plurality of graphical representations are assigned to the left-hand controller and a second portion of the plurality of graphical representations are assigned to the right-hand controller.

10. The surgical robotic system according to claim 7, wherein the controller is further configured to assign each of the instrument movable carts to one graphical representation based on an orientation of the camera.

11. The surgical robotic system according to claim 7, wherein the controller is further configured to assign each of the instrument movable carts to one graphical representation based on an angle of each of the instrument movable carts relative to a surgical table.

12. The surgical robotic system according to claim 7, wherein the display is a touchscreen and each graphical representation of the plurality of graphical representations is movable on the touchscreen.

13. A method for controlling a surgical robotic system, the method comprising:
receiving cart orientation data for each instrument movable cart of a plurality of instrument movable carts;
displaying a graphical user interface having a plurality of graphical representations each of which corresponds to one instrument movable cart; and
assigning each of the instrument movable carts to one graphical representation based on orientation data of the instrument movable carts.

14. The method according to claim 13, further comprising:
assigning a first portion of the plurality of graphical representations to a left-hand controller.

15. The method according to claim 14, further comprising:
assigning a second portion of the plurality of graphical representations to a right-hand controller.

16. The method according to claim 13, further comprising:
receiving camera orientation data for a camera held by a camera movable cart.

17. The method according to claim 16, further comprising:
assigning each of the instrument movable carts to one graphical representation based on the camera orientation data.

18. The method according to claim 17, further comprising:
assigning each of the instrument movable carts to one graphical representation based on an angle of each of the instrument movable carts relative to a surgical table.

19. The method according to claim 13, wherein orientation data of the instrument movable carts includes an angle of each of the instrument movable carts relative to a surgical table.

20. The method according to claim 13, further comprising:
moving at least one graphical representation of the plurality of graphical representations on the graphical user interface.
